(19) United States
(12) Patent Application Publication
(10) Pub. No.: US 2024/0260836 A1
(43) Pub. Date: Aug. 8, 2024

(54) OPTICAL SENSOR CIRCUIT AND OPTICAL SENSING METHOD

(71) Applicant: DeepSight Technology, Inc., Santa Clara, CA (US)

(72) Inventors: DANHAO MA, SAN JOSE, CA (US); LAN YANG, CLAYTON, MO (US); LINHUA XU, UNIVERSITY CITY, MO (US); YIHANG LI, ST. LOUIS, MO (US); DANHUA ZHAO, SAN JOSE, CA (US)

(73) Assignee: DeepSight Technology, Inc., Santa Clara, CA (US)

(21) Appl. No.: 18/429,517

(22) Filed: Feb. 1, 2024

Related U.S. Application Data
(60) Provisional application No. 63/443,105, filed on Feb. 3, 2023.
(60) Provisional application No. 63/592,482, filed on Oct. 23, 2023.
(60) Provisional application No. 63/450,554, filed on Mar. 7, 2023.
(60) Provisional application No. 63/545,327, filed on Oct. 23, 2023.

(51) Int. Cl. A61B 5/00

(52) U.S. Cl. CPC A61B 5/0095

(57) ABSTRACT

The optical sensor circuit is an optical circuit for routing input optical signals through an array of optical sensors. The optical sensor circuit includes an optical input port for receiving a plurality of input optical signals within a single input channel, where each of the input optical signals has a unique wavelength associated therewith. A wavelength-division demultiplexer is coupled to the optical input port to demultiplex the plurality of input optical signals, and a plurality of optical sensors are coupled to the wavelength-division demultiplexer for respectively receiving the plurality of input optical signals and outputting a corresponding plurality of output optical signals. A wavelength-division multiplexer is coupled to the plurality of optical sensors to multiplex the plurality of output optical signals into a single output channel, and an optical output port is coupled to the wavelength-division multiplexer for outputting the plurality of output optical signals in the single output channel.

### CROSS-REFERENCE TO RELATED APPLICATIONS

This application claims the benefit of U.S. Provisional Patent Application No. 63/443,105, filed on Feb. 3, 2023, titled “Optical Sensor Circuit and Optical Sensing Method”; U.S. Provisional Patent Application No. 63/592,482, filed on Oct. 23, 2023, titled “Mixed Transducer Array with Fiber Sensors”; U.S. Provisional Patent Application No. 63/450,554, filed on Mar. 7, 2023, titled “Mixed Array Imaging Probe”; and U.S. Provisional Patent Application No. 63/545,327, filed on Oct. 23, 2023, titled “Miniature Mixed Array Imaging Probe”, each of which is hereby incorporated by reference in its entirety.

### BACKGROUND OF THE INVENTION

### Field of the Invention

The present disclosure relates to optical sensing, and particularly to an optical sensor circuit for routing input optical signals through an array of optical sensors, such as, for example, but not limited to, opto-acoustic sensors or multi-dimensional optical sensors.

### Description of Related Art

Acoustic or ultrasound technology is used in various industries, particularly in non-invasive measurements, remote sensing, and imaging. In medical applications, ultrasound is used in imaging, therapeutic, measurement, sensing and diagnostic procedures. In non-medical applications, ultrasound is used in industrial applications for defect detection, non-destructive testing, structural testing, and microparticle particle sorting, geological applications, including mining and drilling operations, and underwater marine applications, among other applications. Acoustic or ultrasound technology operates by transmitting acoustic signals toward an object and detecting resulting echo signals that reflect or generate from the object in response to the transmitted acoustic signals. Ultrasound is an advantageously non-invasive form of imaging. The resolution of ultrasound increases by transmitting higher frequency acoustic waves. However, the depth of penetration decreases due to the increased acoustic attenuation. This tradeoff between resolution and penetration depth poses a challenge. This invention relates to providing improved ultrasound technology.

### SUMMARY OF THE INVENTION

The optical sensor circuit disclosed herein is an optical circuit for routing input optical signals through an array of optical sensors, such as, for example, but not limited to, opto-acoustic sensors or multi-dimensional optical sensors. The number of optical fibers and input/output (I/O) ports are reduced by transmitting a plurality of input optical signals within a single input channel to an optical input port of an optical sensor circuit. It should be understood that the I/O ports may be on-chip ports, for example, when the optical sensors are integrated within a photonic integrated circuit (PIC) chip, or may be incorporated into any type of optical circuit or optical sensor system.

The optical sensor circuit, in one embodiment, includes an optical input port for receiving a plurality of input optical signals within a single input channel, where each of the input optical signals has a unique wavelength associated therewith. A wavelength-division demultiplexer is coupled to the optical input port to demultiplex the plurality of input optical signals, and a plurality of optical sensors are coupled to the wavelength-division demultiplexer for respectively receiving the plurality of input optical signals and outputting a corresponding plurality of output optical signals. Each of the output optical signals may have a unique wavelength associated therewith matching the wavelength of a corresponding one of the input optical signals. A wavelength-division multiplexer is coupled to the plurality of optical sensors to multiplex the plurality of output optical signals into a single output channel, and an optical output port is coupled to the wavelength-division multiplexer for outputting the plurality of output optical signals in the single output channel. Thus, the number of optical fibers and I/O ports is reduced. As discussed above, it should be understood that the I/O ports may be either on-chip ports or be ports of any other type of suitable optical circuit or optical sensor system.

As a non-limiting example, the plurality of optical sensors may be in the form of a fiber optical sensor array. As a further non-limiting example, the optical sensor circuit may also include an acoustic transducer. As a further non-limiting example, the optical sensor array and the acoustic transducer may be mounted together in a mixed sensor transducer probe.

In an alternative embodiment, a plurality of optical input ports are provided for respectively receiving a plurality of input channels. In this embodiment, each of the input channels carries a plurality of input optical signals, where each of the input optical signals within each of the input channels has a unique wavelength associated therewith. A power splitter is in communication with the plurality of optical input ports for splitting each of the input channels into a plurality of sub-channels carrying a plurality of input optical sub-signals. A wavelength-division demultiplexer is coupled to the power splitter to demultiplex each of the plurality of input optical sub-signals, and a plurality of optical sensors are coupled to the wavelength-division demultiplexer for respectively receiving the plurality of input optical sub-signals and outputting a corresponding plurality of output optical signals. Each of the output optical signals may have a unique wavelength associated therewith matching the wavelength of a corresponding one of the input optical sub-signals. A wavelength-division multiplexer is coupled to the plurality of optical sensors to multiplex the plurality of output optical signals into a plurality of output channels. A plurality of optical output ports are coupled to the wavelength-division multiplexer for outputting the plurality of output optical signals in the plurality of output channels. In this embodiment, a total number of the plurality of output channels is equal to a total number of the output optical signals divided by a total number of the input channels.

Although the wavelength-division demultiplexer, the wavelength-division multiplexer and, in the alternative embodiment described above, the power splitter may all be integrated within the same photonic integrated circuit (PIC) chip as the optical sensors, it should be understood that one or more of these components may be located on a separate optical interposer chip. Thus, rather than coupling the input and output waveguides directly to the PIC chip through the input and output optical ports, the input and output waveguides could be coupled to the interposer chip which, in turn, would be coupled to the input and output ports on the PIC chip. As discussed above, it should be understood that the optical circuit may be integrated into, or include, a PIC chip or the like or, alternatively, it may form, or be part of, any other suitable type of optical circuit or optical sensor system.

The above optical sensor circuits may be integrated into larger optical acoustic sensor systems, and it should be understood that such sensor systems do not necessarily require the components discussed above to be integrated into a PIC chip. In one embodiment, an optical acoustic sensor system includes an acoustic probe, such as an ultrasound probe or the like, for delivering an acoustic signal to a sample to be sensed, such as a tissue sample, a body part, etc. Similar to the previous embodiments, a light source is provided for generating a plurality of input optical signals, where each of the input optical signals has a unique wavelength associated therewith. A wavelength-division demultiplexer is optically coupled to the light source to demultiplex the plurality of input optical signals, and a plurality of optical sensors are provided for sensing the sample. The plurality of optical sensors are optically coupled to the wavelength-division demultiplexer for respectively receiving the plurality of input optical signals and outputting a corresponding plurality of output optical signals. A wavelength-division multiplexer is optically coupled to the plurality of optical sensors to multiplex the plurality of output optical signals into a single output channel. The single output channel is then processed to produce an image of the sample or data representative of sensed physical parameters. The plurality of optical sensors may be provided as an optical sensor array and, further, a heating source may be provided for selectively heating individual ones of the optical sensors of the optical sensor array for tuning thereof.

These and other features of the present subject matter will become readily apparent upon further review of the following specification.

### BRIEF DESCRIPTION OF DRAWINGS

FIG. 1 schematically illustrates an optical sensor circuit.

FIG. 2 schematically illustrates an alternative embodiment of the optical sensor circuit.

FIG. 3 diagrammatically illustrates an optical sensor system.

FIG. 4 schematically illustrates an optical sensor system incorporating the optical sensor circuit of FIG. 1 and/or FIG. 2.

FIG. 5 schematically illustrates an optical acoustic sensor system.

FIG. 6 schematically illustrates an alternative embodiment of the optical acoustic sensor system.

FIG. 7 schematically illustrates an alternative embodiment of the optical sensor circuit.

FIG. 8A and FIG. 8B illustrate a mixed sensor transducer probe.

Similar reference characters denote corresponding features consistently throughout the attached drawings.

### DETAILED DESCRIPTION

Non-limiting examples of various aspects and variations of the invention are described herein and illustrated in the accompanying drawings. The following detailed description is merely illustrative in nature and is not intended to limit the invention or the application and uses of the invention. Although the description of the invention is in the context of optical sensor systems, methods, and devices for ultrasound imaging, the disclosure should not be considered so limiting. For example, although methods may be discussed herein with respect to medical ultrasound, embodiments hereof may be suitable for other medical procedures as well as other procedures or methods in other industries that may benefit from the sensing and imaging technologies described herein. Further, various systems and devices that incorporate optical sensors and photonic integrated sensors are described. It should be understood that optical sensors and photonic integrated sensors, as described herein, may be integrated into and/or used with a variety of systems and devices not described herein. Modifications may be made to the embodiments described herein without departing from the spirit and scope of the present invention. Therefore, the following detailed description is not meant to be limiting. Furthermore, there is no intention to be bound by any expressed or implied theory presented in the preceding technical field, background, summary, or the following detailed description.

Various structures are described herein according to their geometric properties. As discussed herein, all structures so described may vary from the described shape according to the tolerances of known manufacturing techniques. Unless otherwise specified, features described with the term “substantially” are understood to be within 5% of exactness. For example, features described as “substantially parallel” may deviate from true parallel by 5%.

Some existing ultrasound technologies use acoustic energy generating (AEG) materials for transducers to generate and receive acoustic signals. Commonly used AEG transducers include piezoelectric materials such as lead-zirconate-titanate (PZT), ceramic, piezoelectric single crystal (e.g., PIN-PT, PIN-PMN-PT), polymer thick film (PTF), polyvinylidene fluoride (PVDF), capacitive micromachined ultrasonic transducers (CMUT), photoacoustic transducers, piezoelectric micromachined ultrasound transducers (PMUT), among many other materials known to those of skill in the art. However, some of the challenges associated with use of these materials aside from the trade-offs between resolution and penetration depth, include high operation voltage requirements, a high electric field requirement (which may cause breakdown and failure), a non-linear response with high hysteresis, and limited angle of detection. Furthermore, the detection sensitivity of AEG transducers is a function of size, thereby limiting the suitability of size-constrained applications such as intravascular ultrasound (IVUS) devices.

Another challenge is the narrow bandwidth of an AEG transducer. For example, for ultrasound transducers made of piezoelectric material, such as lead zirconate titanate (PZT), the 6 dB bandwidth of PZT is generally limited to only about 70%. Certain composite PZT materials have a slightly increased bandwidth, but still only achieve a bandwidth of up to about 80%. As another example, single crystal materials have increasingly been used in an effort to improve performance of ultrasound probes but have lower Curie temperatures and are brittle. Another type of transducer material is silicon, which can be processed to build Capacitive Micromachined Ultrasound Transducer (CMUT) probes that can have increased bandwidth. However, CMUT probes are not very sensitive or reliable. Moreover, CMUT probes have several operational limitations. For example, CMUT probes are nonlinear transducers and, therefore, are not generally suitable for harmonic imaging. In addition, CMUT probes require an additional bias voltage to operate properly. Thus, there is a need for new and improved devices and methods for ultrasound imaging modes with various frequency harmonics to obtain higher resolution, better penetration, and fewer artifacts than fundamental imaging of conventional ultrasound sensing.

In many applications, it is desirable to detect multiple kinds of physical parameters. For example, in the field of medical technology, it may be advantageous to have medical devices with sensors that can sense multiple different physical parameters (e.g., simultaneously in real-time or near real-time). For example, ablation catheters for cardiovascular procedures may include temperature sensors to measure the temperature of the treated tissues and force sensors to measure the force applied to the arterial wall during heart ablation. It may be possible to incorporate multiple kinds of sensors together in a single device to monitor multiple different kinds of parameters, in addition to, or instead of, imaging. However, the inclusion of more sensors may result in a device that may be more challenging to fit into a desired form factor. Additionally or alternatively, the inclusion of more sensors may pose more difficulties in accommodating additional components (e.g., mechanical and/or electrical) and connections to enable proper functioning of all of the different sensors.

The use of optical sensors as multi-dimensional sensors for sensing physical parameters alleviates many difficulties associated with combining multiple sensors and their various components and connections. To accomplish multi-dimensional sensing, measurement signals are generated from optical sensor responses, where each of these measurement signals may be indicative of a respective physical signal. For example, a signal processor may generate a temperature measurement signal based at least in part on the resonant frequency shift (e.g., mode shift) and an acoustic measurement signal based at least in part on oscillation of optical power. Multi-dimensional sensing can also be achieved by using multiple sensors, each responding differently to different sensing targets. Variations of generating measurement signals from optical sensor responses, may include decoupling individual physical signals and/or collectively analyzing the multiple sensor responses to determine individual physical signals.

Photonic devices and optical pressure detection techniques have shown great promise for ultrasound detection. In photonic devices, refractive index modulation and/or shape deformations due to strain induced by an acoustic wave are translated into changes in the intensity of the detected light or the spectral properties of the device. In some existing devices, optical resonators have been used as highly sensitive ultrasound detectors. In general, the performance of an optical resonator is limited by its quality factor Q (i.e., the higher the Q, the lower the optical loss and the smaller the detectable resonance shift) as well as by the acousto-optical and mechanical properties of the material from which the resonator is made. Optical sensors, such as, for example, whispering galley mode (WGM) optical resonators, may have high sensitivity and broad bandwidth in reception of ultrasound signals compared to other types of ultrasound sensors. Because of the high sensitivity and broad bandwidth of optical sensors, the image produced by the optical sensors may have improved spatial resolution, improved penetration depth, improved signal-to-noise ratio (SNR), improved tissue harmonic imaging, and/or improved Doppler sensitivity.

Acousto-optic systems based on optical sensors may directly measure ultrasonic waves through the photo-elastic effect and/or physical deformation of the resonator(s) in response to the ultrasonic waves (e.g., ultrasonic echoes). For example, in the presence of ultrasonic (or any pressure) waves, the WGMs traveling an optical resonator may undergo a spectral shift caused by changes in the refractive index and shape of the optical resonator. The spectral change can be easily monitored and analyzed in spectral domain and light transmission intensity to and from the optical resonator. Additional spatial and other information can furthermore be derived by monitoring and analyzing shifting WGMs among multiple optical resonators.

Optical sensors described herein may include an interference-based optical sensor(s), such as an optical resonator(s), an optical interferometer(s), etc. The optical resonators may include, for example, a whispering galley mode (WGM) optical resonator(s), a microbubble optical resonator(s), a microsphere resonator(s), a micro-toroid resonator(s), a micro-ring resonator(s), a micro-disk optical resonator(s), and/or the like. The optical interferometers may include a Mach-Zehnder interferometer(s), a Michelson interferometer(s), a Fabry-Perot interferometer(s), a Sagnac interferometer(s), and/or the like. For example, a Mach-Zehnder interferometer may include two nearly identical optical paths (e.g., fibers, on-chip silicon waveguides, etc.). The two optical paths may be finely adjusted acoustic waves (e.g., by physical movement caused by the acoustic waves, tuning of refractive index caused by the acoustic waves, etc.) to effect distribution of optical powers in an output(s) of the Mach-Zehnder interferometer, and therefore, detect a presence or a magnitude of the acoustic waves.

The optical resonators may include a closed loop of a transparent medium that allows some permitted frequencies of light to continuously propagate inside the closed loop, and to store optical energy of the permitted frequencies of light in the closed loop. For example, the optical resonators may permit propagation of whispering gallery modes (WGMs) traveling the concave surface of the optical resonators and corresponding to the permitted frequencies to circulate the circumference of the resonator. Each mode from the WGMs corresponds to propagation of a frequency of light from the permitted frequencies of light. The permitted frequencies of light and the quality factor of the optical resonators described herein may be based at least in part on geometrical parameters of the optical resonator, refractive index of the transparent medium, and refractive indices of an environment surrounding the optical resonator.

Acoustic or ultrasound capabilities can be categorized with respect to sensitivity, resolution, and field of view, among others. Sensitivity is related to the single sensor element design and optimization. Resolution and field of view are limited by sensor array configuration, which includes space between adjacent sensors, total number of sensors in one imaging probe, length and width of sensor arrays covering a sufficient field of view, etc. Thus, challenges exist in designing a robust efficient acoustic-optical sensor with minimal loss in the form factor needed.

Several factors must be considered when designing optical sensors for physical measurements and/or acoustic detection. Optical loss must be minimized, as loss will fundamentally affect the efficiency of the sensor. Careful attention is needed when designing the coupling gap between the resonator and waveguide, along with determining the appropriate waveguide cross section. Noise must be minimized to optimize the SNR, and various parameters must be balanced, such as laser power, the array size required, the space available on a chip or array structure, the power required, the number of channels needed in view of the number of sensors needed, and the effect of all components along the signal path.

As will be discussed in detail below, the optical sensor circuit disclosed herein is an optical circuit for routing input optical signals through an array of optical sensors, such as, for example, but not limited to, opto-acoustic sensors and/or optical sensors for sensing physical parameters. The number of optical fibers and on-chip or fiber sensor input/output (I/O) ports (cables) are reduced by transmitting a plurality of input optical signals within a single input channel to an optical input port of an optical sensor circuit. As discussed above, it should be understood that the optical circuit may be integrated into, or include, a PIC chip or the like or, alternatively, it may form, or be part of, any other suitable type of optical circuit or optical sensor system.

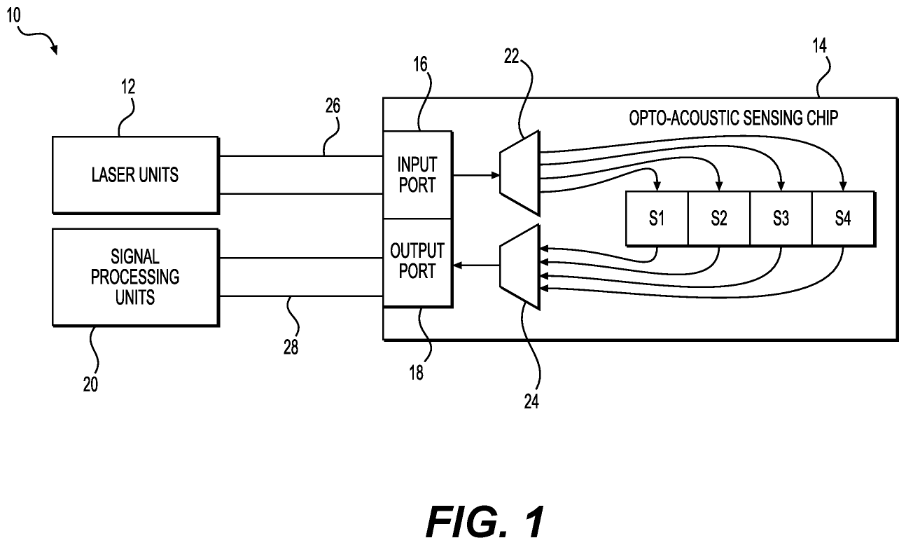

As shown in FIG. 1, in one embodiment, the optical sensor circuit 10 is an optical circuit for routing input optical signals through an array of optical sensors, such as, for example, but not limited to, opto-acoustic sensors and/or optical sensors for sensing physical parameters. As shown in FIG. 1, the optical sensor circuit 10 includes an optical input port 16 for receiving a plurality of input optical signals within a single input channel, where each of the input optical signals has a unique wavelength associated therewith. As a non-limiting example, four laser units 12 may be used to deliver four optical signals at unique wavelengths λ₁, λ₂, λ₃ and λ₄ through a single optical fiber 26 connected to a single input port 16 on a photonic integrated circuit (PIC) chip 14. Four optical signals are transmitted through a single optical fiber to a single input port, in this example, reducing the number of optical fibers and I/O ports needed. It should be understood that any suitable number of optical signals with unique wavelengths may be used, and that the four signals discussed above are a non-limiting example only. Additionally, it should be understood that any suitable type of lasers may be used to generate the input optical signals, and that any suitable type of optical fiber and optical input port may be used. Further, it should be understood that single optical fiber 26 is shown for exemplary purposes only, and that any suitable type of optical waveguide may be utilized. Additionally, as discussed above, it should be understood that the optical circuit may be integrated into, or include, a PIC chip or the like or, alternatively, it may form, or be part of, any other suitable type of optical circuit or optical sensor system.

The PIC chip 14, in this non-limiting example, includes a wavelength-division demultiplexer 22 coupled to the optical input port 16 to demultiplex the plurality of input optical signals, and a plurality of optical sensors S1, S2, S3 and S4 are coupled to the wavelength-division demultiplexer 22 for respectively receiving the plurality of input optical signals and outputting a corresponding plurality of output optical signals. Following the non-limiting example discussed above, the wavelength-division demultiplexer 22 separates the four optical signals with respective wavelengths of λ₁, λ₂, λ₃ and λ₄, which were transmitted through a single channel via single optical fiber 26, and these individual optical signals are respectively input to optical sensors S1, S2, S3 and S4, which are also included on PIC chip 14. It should be understood that sensors S1, S2, S3 and S4 may be any suitable type of optical sensors, such as, but not limited to, opto-acoustic sensors and/or multi-dimensional optical sensors. Further, it should be understood that any suitable type of optical fibers, waveguides or the like may be used to couple wavelength-division demultiplexer 22 with the array of sensors S1, S2, S3 and S4.

The output of each sensor S1, S2, S3 and S4 typically has the same wavelength as the input signal; i.e., the optical signals output from the array of sensors S1, S2, S3 and S4 in the present non-limiting example will typically have respective wavelengths of λ₁, λ₂, λ₃ and λ₄. As a non-limiting example, if sensors S1, S2, S3 and S4 are opto-acoustic sensors, then the output of each sensor may be an optical signal having the same wavelength as the input signal, but with a shifted phase, and this shifted phase would represent an acoustic force sensed by sensors S1, S2, S3 and S4. As another non-limiting example, if sensors S1, S2, S3 and S4 are multi-dimensional sensors, a signal processor may generate a measurement signal, such as temperature, based at least in part on the resonant frequency shift (e.g., mode shift) and an acoustic measurement signal based at least in part on oscillation of optical power.

A wavelength-division multiplexer 24 is coupled to the plurality of optical sensors S1, S2, S3 and S4 to multiplex the plurality of output optical signals into a single output channel. It should be understood that any suitable type of optical fibers, waveguides or the like may be used to couple wavelength-division multiplexer 24 with the array of sensors S1, S2, S3 and S4. An optical output port 18 is coupled to the wavelength-division multiplexer 24 for outputting the plurality of output optical signals in the single output channel via a single optical fiber 28, which may then be coupled to one or more signal processing units 20 for performing any necessary signal processing dependent upon the particular type of sensors S1, S2, S3 and S4, and the particular application of PIC chip 14. It should be understood that single optical fiber 28 is shown for exemplary purposes only, and that any suitable type of optical waveguide may be utilized.

The usage of demultiplexer 22 and multiplexer 24 allows the number of input and output ports 16, 18, respectively, to be reduced. Particularly, the number of input and output ports 16, 18 is reduced by the wavelength-division multiplexer (WDM) channel number, w, where w represents the number of different wavelengths involved. In the above non-limiting example, w is 4. It should be understood that any suitable type of multiplexing/demultiplexing devices may be used. Non-limiting examples of such devices which are suitable for integration on PIC chips include arrayed waveguide gratings (AWGs), Echelle gratings, Mach-Zehnder interferometers (MZIs), and inverse-designed wavelength (de)multiplexers. Due to the requirement of pairing with a dense array of sensing units, multiplexing/demultiplexing devices with compact footprints are preferred. In particular, the outline of demultiplexer 22 and/or multiplexer 24 should be compact, at least in the lateral dimension of the sensor array where a small pitch is required. The size of the demultiplexer 22 and/or multiplexer 24 outline in the lateral dimension should be maintained at least under pitch×w. It should be understood that input and output ports 16, 18 may be any suitable type of optical couplers. As a non-limiting example, input and output ports 16, 18 may be, or include therein, spot size converters (SSCs).

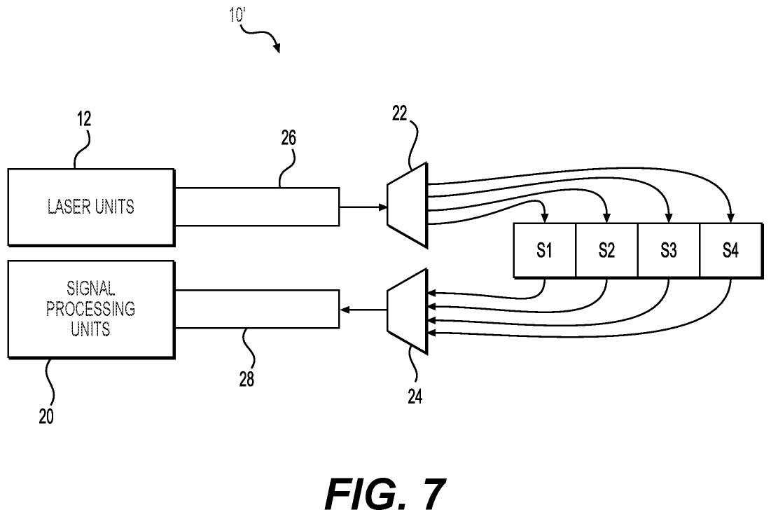

As discussed above, it should be understood that the optical circuit does not necessarily have to include a PIC chip. As a non-limiting example, as shown in FIG. 7, optical circuit 10′ is substantially identical to optical circuit 10, however, PIC chip 14 has been removed, and the demultiplexer 22, the multiplexer 24 and the plurality of optical sensors S1, S2, S3 and S4 may be mounted on any suitable structure(s) or housings, or may be incorporated into any associated device(s) including those for acoustic sensing and/or for multi-dimensional signal detection.

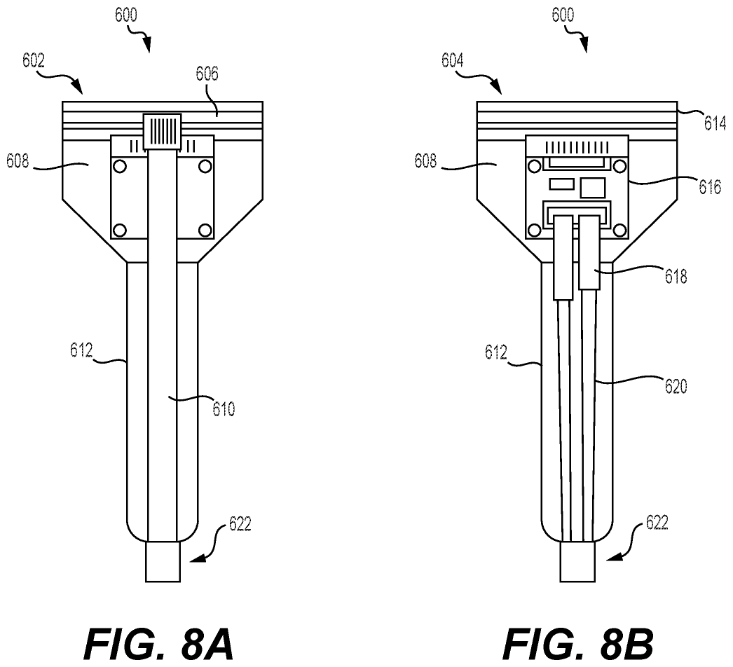

As a further non-limiting example, the plurality of optical sensors and an acoustic generator may be housed together in a probe. In the non-limiting example of FIGS. 8A and 8B, the plurality of optical sensors and an acoustic generator are housed together in a mixed sensor transducer probe 600. The mixed sensor transducer probe 600 may be suitable for external use. As a further non-limiting example, the plurality of optical sensors may be provided in the form of an optical acoustic transducer 602, and the acoustic transducer may be an acoustic energy generator (AEG) based transducer 604. These two components are illustrated in FIGS. 8A and 8B as separate portions for ease of discussion. It should be understood that the features of these two components may be mixed and intermingled as necessary for functionality, as discussed in greater detail below.

The optical acoustic transducer 602 illustrated in FIG. 8A may include, as a non-limiting example, an optical sensor array 606 including one or more optical sensors contained within a probe head 608 of the mixed sensor transducer probe 600. The optical acoustic transducer 602 may further include an optical waveguide 610 (e.g., a fiber optic cable) disposed within a handle 612 of the mixed sensor transducer. The AEG based transducer 604 illustrated in FIG. 8B may include an AEG transducer stack 614 including one or more AEG transducers and components necessary for their operation contained within a probe head 608 of the mixed sensor transducer 600. The AEG based transducer 604 may further include a circuit 616, such as a flex circuit or the like, an interconnect 618, and a connection cable 620 (e.g., a coaxial cable or the like). These may be disposed within a handle 612 of the mixed sensor transducer 600 and/or within the probe head 608, as necessary. The mixed sensor transducer 600 may further include a mixed cable 622 configured to carry both the optical waveguide 610 and the connection cables 620 back to a system.

The optical sensor array 606 may include a bundle of fiber optical sensors or, in a further non-limiting example, the optical sensor array 606 may include an on-chip optical sensor array. Additionally, the demultiplexer 22, the multiplexer 24, and any suitable ports, couplings or other types of connections, interconnections or the like may also be received within the handle 612. It should be understood that the overall configuration, shape and relative dimensions of the mixed sensor transducer probe 600 are shown for exemplary purposes only and may be varied. Further, it should be understood that at least a portion of the mixed sensor transducer probe 600 may be incorporated into other structures or devices. As a non-limiting example, handle 612 may include, be attached to, incorporate or be incorporated by a catheter or similar structure.

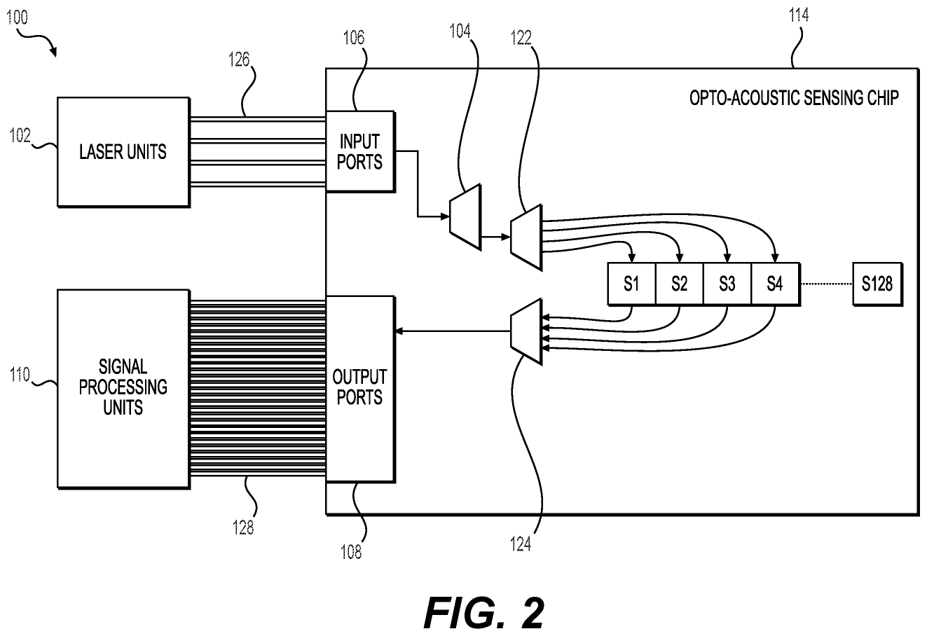

In the alternative embodiment of FIG. 2, the optical sensor circuit 100 includes a plurality of optical input ports 106 on PIC chip 114. The plurality of optical input ports 106 are provided for respectively receiving a plurality of input channels via a plurality of optical fibers 126. As a non-limiting example, four lasers 102 may be used to generate optical signals with unique wavelengths λ₁, λ₂, λ₃ and λ₄. A bundle of four optical fibers 126 couples laser units 102 to input ports 106 of PIC chip 114. Each individual optical fiber carries four optical signals with wavelengths of λ₁, λ₂, λ₃ and λ₄. Thus, over four channels, there are 16 different optical signals, with four signals per channel in this non-limiting example. It should be understood that optical fibers 126 are shown for exemplary purposes only, and that any suitable type of optical waveguides may be utilized. Further, as discussed above, it should be understood that the optical circuit may be integrated into, or include, a PIC chip or the like or, alternatively, it may form, or be part of, any other suitable type of optical circuit or optical sensor system.

In this non-limiting example, PIC chip 114 may optionally, be provided with a power splitter 104, which is in communication with the plurality of optical input ports 106 for splitting each of the input channels into a plurality of sub-channels carrying a plurality of input optical sub-signals. It should be understood that any suitable type of optical power splitter may be used. Non-limiting examples of power splitters which may be utilized include Y-branch optical waveguides and multi-mode interferometers. Continuing the non-limiting example discussed above, if power splitter 104 is a 1×8 splitter (i.e., dividing each optical signal into eight optical sub-signals, each with a fraction of the power of the original signal), then 128 separate optical sub-signals will be generated by power splitter 104; i.e., 16 optical signals split 8 times, resulting in 128 sub-signals. It should be understood that any suitable type of optical fibers, waveguides or the like may be used to couple power splitter 104 with the plurality of input ports 106. Power splitter 104 may evenly split the overall power/intensity of each optical signal; i.e., in the example discussed above, each of the eight optical sub-signals may have ⅛ the power/intensity of the original optical signal.

A wavelength-division demultiplexer 122 is coupled to the power splitter 104 to demultiplex each of the plurality of input optical sub-signals, and a plurality of optical sensors are coupled to the wavelength-division demultiplexer 122 for respectively receiving the plurality of input optical sub-signals and outputting a corresponding plurality of output optical signals. Following the non-limiting example discussed above, the wavelength-division demultiplexer 122 separates the 128 optical sub-signals, each of which has a wavelength of λ₁, λ₂, λ₃ or λ₄, and these individual optical sub-signals are respectively input to optical sensors S1, S2, S3, S4, . . . , S128, which are also included on PIC chip 114. It should be understood that sensors S1, S2, S3, S4, . . . , S128 may be any suitable type of optical sensors, such as, but not limited to, opto-acoustic sensors. Further, it should be understood that any suitable type of optical fibers, waveguides or the like may be used to couple wavelength-division demultiplexer 122 with the array of sensors S1, S2, S3, S4, . . . , S128.

The output of each sensor S1, S2, S3, S4, . . . , S128 typically has the same wavelength as the input sub-signal; i.e., the optical signals output from the array of sensors S1, S2, S3, S4, . . . , S128 in the present non-limiting example will typically have a wavelength of λ₁, λ₂, λ₃ or λ₄, matching the wavelength of the corresponding input optical sub-signal. A wavelength-division multiplexer 124 is coupled to the plurality of optical sensors S1, S2, S3, S4, . . . , S128 to multiplex the plurality of output optical signals into a plurality of output channels. Each output channel contains optical signals with the same wavelengths contained in each of the input channels; i.e., continuing the present non-limiting example, the wavelength-division multiplexer 124 receives the 128 output optical signals from the optical sensors S1, S2, S3, S4, . . . , S128 and outputs 32 optical channels, where each channel contains four signals with respective wavelengths of λ₁, λ₂, λ₃ or λ₄. A plurality of optical output ports 108 on PIC chip 114 are coupled to the wavelength-division multiplexer 124 for outputting the plurality of output optical signals in the plurality of output channels. In general, a total number of the plurality of optical output ports 108 (which is equal to the total number of output channels) is equal to a total number of the output optical signals divided by a total number of the input channels. Thus, in this non-limiting example, 32 output ports 108 are provided for coupling the PIC chip 114 to signal processing units 110 via 32 optical fibers 128.

It should be understood that optical fibers 128 are shown for exemplary purposes only, and that any suitable type of optical waveguides may be utilized. It should be further understood that input and output ports 126, 128 may be any suitable type of optical couplers. As a non-limiting example, input and output ports 126, 128 may be, or include therein, spot size converters (SSCs). It should be further understood that the four input optical sub-signals and four output optical signals shown in FIG. 2 are for purposes of simplification and illustration only, and that each of sensors S1, S2, S3, S4, . . . , S128 will have an input optical sub-signal from wavelength-division demultiplexer 122 and a corresponding number of output optical signals.

Although the wavelength-division demultiplexer 22/122, the wavelength-division multiplexer 24/124 and, in the alternative circuit 100, the power splitter 104 may all be integrated within the same photonic integrated circuit (PIC) chip 14/114 as the optical sensors, it should be understood that one or more of these components may be located on a separate optical interposer chip. Thus, rather than coupling the input and output waveguides directly to the PIC chip through the input and output optical ports, the input and output waveguides could be coupled to the interposer chip which, in turn, would be coupled to the input and output ports on the PIC chip 14/114. In practice, the materials and fabrication process for the PIC chip do not have a large variable range, since the particular sensing units integrated on the PIC chip often require very specific materials with very specific device parameters. By moving demultiplexer 22/122, multiplexer 24/124 and/or power splitter 104 to a separate interposer chip, the selection of the materials and fabrication processes would be greatly expanded since the requirements specific to the sensing units would not apply to the interposer chip.

Considering an example in which only the power splitter 104 is moved to a separate interposer chip coupled to the PIC chip, a material with a higher power damage threshold, such as silicon nitride, could be used to manufacture the interposer chip, while silicon would still be used to manufacture the PIC chip. The ability to use silicon nitride in the interposer chip in this example would solve the power budget problem of power splitting. It should be understood that silicon and silicon nitride, respectively, are non-limiting examples discussed for purposes of illustration only. Overall, the division of components between the PIC chip and an additional interposer enables more variety in structure, configuration and design, particularly on the interposer chip, thus enabling optimization of coupling efficiency. The addition of an interposer allows, as a non-limiting example, the usage of specific suspended, high-power damage threshold material to be used, such as the silicon nitride discussed in the above example. As a further non-limiting example, multi-layer couplers could be incorporated into the interposer. Both of these non-limiting examples could affect the overall yield and/or be incompatible with the sensor chip design and fabrication process, as well as increasing wafer costs, thus it is not feasible or practical to make use of them in PIC chips, but by adding the interposer chip, these materials and designs can be used without interfering with the primary sensor PIC chip.

With regard to the present optical sensor circuits 10, 100, it is noted that WDM branching adds to the power budget; i.e., the shared input waveguide needs to handle the power of p×w channels, where p is the power split ratio and w is the WDM channel number. By transferring the power splitter 104 to the interposer chip, a higher power splitting ratio can be achieved. It is noted that although the number of the input ports 16/106 on the PIC chip 14/114 will increase if the power splitter 104 is moved to the interposer chip, the total width of the input ports 16/106 (and thus the width of PIC chip 14/114) can be maintained or even reduced, since the pitch of the couplers on the interposer chip is not limited by the fiber diameter. Additionally, the fabrication flexibility of the interposer chip allows for special chip couplers which can increase the coupling efficiency with optical fibers and sensor chips. Thus, a lower insertion loss of the optical I/O can be achieved, even with more coupling interfaces brought by the interposer.

It should be understood that, similar to the previous embodiment, the optical sensors may be provided as, for example, an optical sensor array incorporated into a mixed sensor transducer probe, such as that shown in FIGS. 8A and 8B and described above. Similarly, the demultiplexer 122, the multiplexer 124, the power splitter 104, and any suitable ports, couplings or other types of connections, interconnections or the like may also be received within the handle 612 of mixed sensor transducer probe 600 or a similar structure or device.

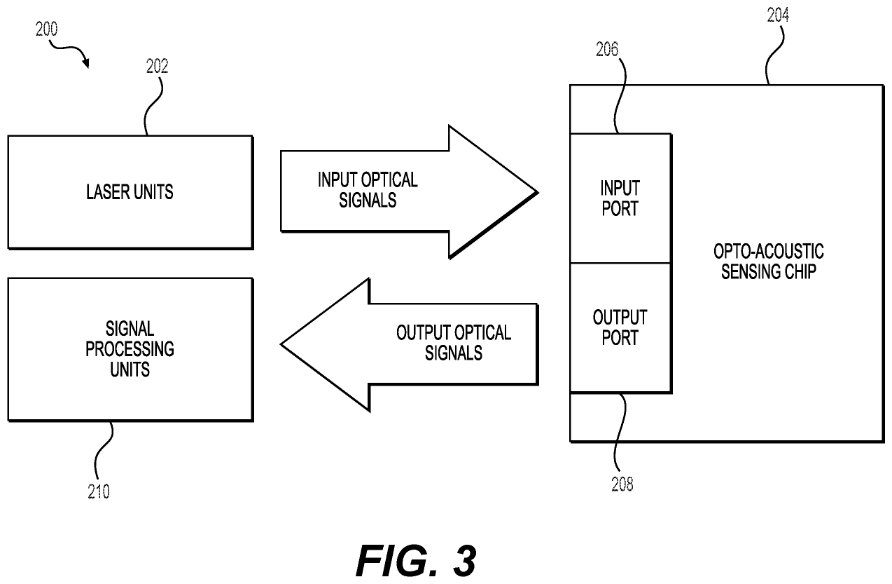

In general, as illustrated in FIG. 3, an opto-acoustic sensor system 200 operates by pumping a light signal at a fixed optical wavelength into an on-chip sensing unit contained within a photonic integrated circuit (PIC) chip 204. One or more light signals are typically generated by one or more laser units 202 such that each signal has a known and constant wavelength. Within the chip 204, the light signal responds to acoustic/ultrasound pressure scattered from an imaged object or patient. Output light signals from an array of sensors on the PIC chip 204 are then transmitted off the chip 204 into signal processing units 210 for, for example, bio-medical imaging generation or providing information or data related to sensed physical parameters. In an opto-acoustic sensor system, the light signals generated by laser units 202 passing through the sensor arrays are modified by acoustic/ultrasound pressure waves, resulting in output light signals which are changed by this modification. These changes (in phase, for example) allow the system to derive information regarding the target(s). In multi-dimensional sensing, measurement signals indicative of a respective physical parameters are generated from the modified light signals.

The sensitivity of system 200 is closely related to the amount of light available in the on-chip sensing unit. While the sensing performance usually benefits from higher light intensity (i.e., optical power) per channel, numerous different designs and optimization schemes have been utilized to maximize the efficiency of the sensing units. Laser units 202 have output power that is limited by commercial availability and is typically provided in the range of 1 mW to 200 mW, which is sufficiently high to support multiple well-designed sensing units. The number of sensing units that can be optically powered by one laser unit may be defined by a power split ratio, p. In practice, the light signals are transmitted into the sensor units via an on-chip optical I/O interface consisting of one or more optical input ports 206 and one or more optical output ports 208. This interface couples light from input optical waveguides, which may be optical fibers, for example, on the laser side into on-chip waveguides on the sensing unit side.

After the light is transmitted onto the sensor chip 204, the light signals may be evenly separated into multiple channels before being fed into individual sensing units. As will be described in more detail below, this power splitting is performed to allow the use of high output power laser units, which subsequently minimizes the total number of costly laser units 202 required. After opto-acoustic sensing, the output light signals are transferred into off-chip analysis units via the one or more optical output ports 208. This output interface couples light from waveguides on the sensing unit side to output optical waveguides (e.g., optical fibers) on the analysis unit side.

Conventionally, each light signal from the sensing unit gets transmitted via its individual optical I/O interface into its corresponding signal analysis unit 210. If an imaging probe, for example, contains N sensor units, then a total of N/p input optical waveguides and input ports are required, and N output optical waveguides are required (i.e., 1+1/p optical ports per channel). For example, if an array of 128 individual sensors are contained in chip 204, then 128 output optical fibers are required. All of the input and output optical fibers are connected to input and output ports 106, 108, respectively. For an imaging configuration of 128 sensing units, 144 total optical ports and optical fibers are required. This is based on a sensor chip that separates the optical input signal into 8 channels (due to sensor requirements), thus requiring 16 optical input ports and 128 optical output ports, totaling 144 ports overall.

As imaging resolution and field of view increase, the number of input/output optical waveguides and on-chip optical I/O ports increase with the number of sensing elements. Manufacturing a single imaging probe that interfaces with 100 optical fibers, as an example, is both difficult and expensive. The difficulty largely arises from the need for optical alignment at the optical I/O interface to ensure all of the optical fibers are aligned accurately and fixed permanently to their corresponding on-chip optical ports. The raw material cost is large due to the large number of optical fibers, and the operational cost for manufacturing such a chip is even larger. In addition to the above, as a practical example, the alignment of a fiber-edge coupler on a PIC chip requires accuracy on the order of a micrometer since the mode profile of a conventional fiber is only 10 μm in diameter. Thus, for more than 100 optical channels with an approximately 0.127 mm pitch, the width of a fiber array unit must be more than 10 mm, requiring an angle tolerance of ˜ 1/10,000 rad. In practice, the inevitable bending of the fiber array unit may even make such an attachment impossible. Thus, it is very important to the optical sensing industry to be able to significantly reduce the number of optical fibers and on-chip I/O ports, thus enabling the expansion of on-chip sensing arrays for higher resolution and wider fields of view.

Additionally, the resolution and field of view requirements for imaging may generate criteria for the form factor of the imaging probe. A common criterion is the pitch of a linear sensor array, which typically ranges from 0.1 mm to 0.3 mm. A typical optical fiber has a diameter of 0.125 mm. Thus, the reduction of optical ports per channel is important to avoid significant changes in the form factor of the probe front end due to the spatial requirement of optical I/Os.

As discussed above, one method of reducing the number of optical fibers and on-chip I/O ports is optical power branching; i.e., where a single optical signal is divided or split into multiple signals, each at a fraction of the original power, thus reducing the number of input optical ports. After light is transmitted onto the sensor chip, the light signals undergo a 1-to-p power splitting before being fed into the individual sensor units. For a light source at a fixed wavelength, the laser light power can be increased so that only one input optical fiber and an on-chip input port is needed to support all of the light power requirements for all on-chip sensing elements. As an example, for a chip with optical power branching of 1×8 (i.e., one signal split into eight signals of ⅛ power of the original), one laser source at a fixed wavelength could support eight sensing elements via one on-chip input port and a single optical splitter. Although the benefits of such a theoretical scenario are clear, such an approach is impractical due to the limited accessibility and safety concerns of the high-power laser sources which would be required, as well as the power budget of the on-chip port and waveguide before the power splitting. Additionally, this technique still requires a large number of optical fibers and on-chip I/O ports for the transmission of the output signals. Thus, it would clearly be desirable to be able to make use of an alternative, more efficient form of splitting, either on its own or in combination with power splitting. However, as discussed above, it should be understood that the optical circuit may be integrated into, or include, a PIC chip or the like or, alternatively, it may form, or be part of, any other suitable type of optical circuit or optical sensor system.

It should be understood that the optical sensor circuits 10, 100 may be incorporated into any suitable type of sensor system. As a non-limiting example, the sensor system may include a fiber optical sensor array or mixed sensor transducer that can be incorporated in an ex vivo or in vivo device, such as for imaging, diagnostic procedures, therapeutic procedures, multi-dimensional sensing, object visualization or tracking, ultrasound, interoperative ultrasound, endoluminal ultrasound (EUS), endobronchial ultrasound (EBUS) or intravascular ultrasound (IVUS). As non-limiting examples, the optical sensors may be any one of, or be similar to, the optical sensors described in the following co-pending applications: U.S. patent application Ser. No. 17/832,507, titled “Whispering Gallery Mode Resonators for Sensing Applications”; U.S. application Ser. No. 17/956,640, titled “Optical Microresonator Array Device”; and International Patent Application No. PCT/US2022/04125, titled “Multi-dimensional Signal Detection with Optical Sensor”, each of which is hereby incorporated by reference in its entirety. The ex vivo or in vivo device may be one of, or be similar to, any of the mixed arrays described in the following co-pending applications: U.S. patent application Ser. No. 17/990,596 titled “Mixed Ultrasound Transducer Arrays”; U.S. patent application Ser. No. 17/244,605 titled “Modularized Acoustic Probe”; and International Patent Application No. PCT/US2022/077762, filed on Oct. 7, 2022, titled “Ultrasound Beacon Visualization with Optical Sensors”, each of which is hereby incorporated herein by reference in its entirety.

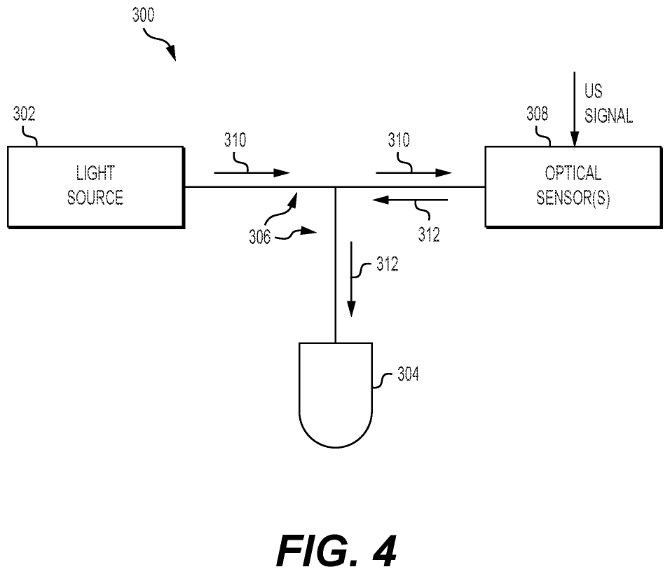

As a non-limiting example, FIG. 4 illustrates an optical sensor system 300 for use with optical sensors adapted and/or configured to detect acoustic signals. The optical sensor system 300 includes a light source 302, such as a laser, a light reception device 304, such as a photodetector or the like, one or more optical waveguides 306, and optical sensors 308, which may be, or incorporate therein, optical sensor circuit 10 and/or 100. In operation, the light source 302 supplies the initial optical signal 310 to the optical sensors 308 via the optical waveguides 306. The supplied initial optical signal 310 is returned by the optical sensors 308 back along the optical waveguide 306. The returned optical signal 312 travels via the optical waveguides 306 and is received at the light reception device 304. As discussed above, acoustic signals, such as ultrasound (US) signals, for example, incident on the optical sensors 308 alter the optical characteristics (which may include the physical structure as well as the optical material properties) of the optical sensors 308. Such optical characteristic alterations may be measured according to changes in the returned optical signal 312.

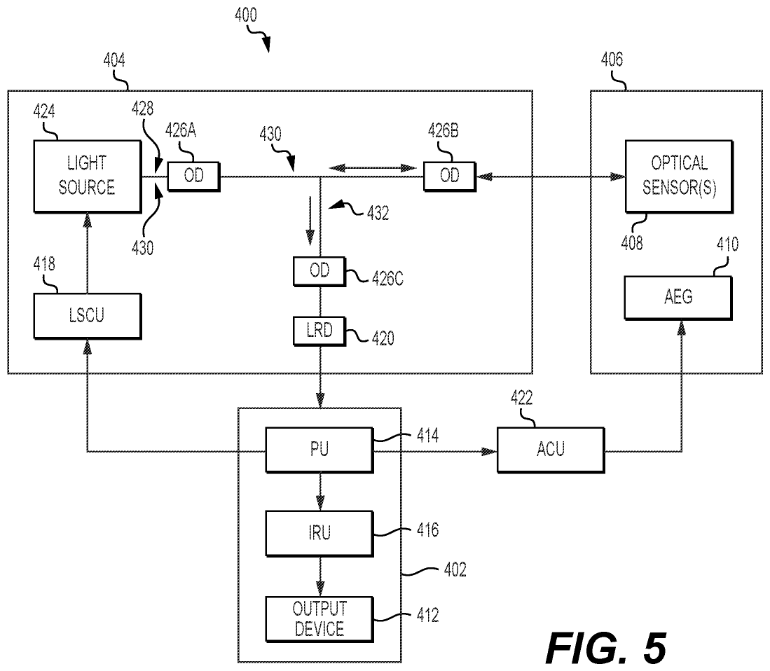

In the further embodiment of FIG. 5, an optical acoustic sensor system 400 is provided. It should be understood that optical acoustic sensor system 400 may include any suitable type of hardware and components to facilitate the use of an ultrasound transducer and/or ultrasound probe. The optical acoustic sensor system 400 may include a processing system 402, an optical sub-system 404, and a transducer probe 406 that includes optical sensors 408, and an AEG transducer 410. Optical sensors 408 may be any suitable type of optical sensors, such as, but not limited to, opto-acoustic sensors. The optical acoustic sensor system 400 includes components, devices, hardware and software to facilitate the use of optical sensors 408. The optical sensors 408 may include a fiber sensor array, a photonic integrated sensor array, or any other suitable sensor arrangement. The AEG transducer 410 may be used for generating and receiving acoustic signals or just generating acoustic signals.

The processing system 402 may include a processing unit (PU) 414 and an image reconstruction unit (IRU) 416. Processing unit 414 may include at least one computer processor, at least one non-transitory computer readable storage medium, and appropriate software instructions. The processing unit 414 is configured to provide control signals to, and receive information signals from, a light source control unit (LSCU) 418, a light receiving device (LRD) 420, and an acoustic control unit (ACU) 422. The processing unit 414 may communicate (via control signals and information signals) with the light source control unit 418, thereby providing control of optical signals provided to the optical sensors 408. The processing unit 414 may communicate (via control signals and information signals) with the acoustic control unit (ACU) 422, thereby providing control and reception of acoustic signals via the acoustic probe 406. The processing unit 414 is further configured to communicate with the light receiving device 420 to receive information signals associated with optical signals received by the light receiving device 420. Thus, processing unit 414 operates to provide the necessary control signals and receive the acquired information signals in the optical acoustic sensor system 400.

The processing unit 414 is further in communication with the image reconstruction unit 416, which operates to generate images based on the data and/or information acquired by the processing unit 414. The image reconstruction unit 416 may generate images based on data related to a medium, such as a human body, captured by the optical sensors 408 and the AEG probe 410. The image reconstruction unit 416 may be integrated within a system containing the processing unit 414 and/or may be a separate system including at least one computer processor, at least one non-transitory computer readable storage medium, and appropriate software instructions. The processing system 402 may provide control signals to the output device 412 to provide a data output. The output device 412 may include, for example, a display or a device including a display. In some variations, the system 400 may further include a set of ancillary interface devices (not shown) used to input information to the system 400 or output information from system 400. The set of ancillary devices may include, for example, a keyboard(s), a mouse(s), a monitor(s), a webcam(s), a microphone(s), a touch screen(s), a printer(s), a scanner(s), a virtual reality (VR) head-mounted display, a joystick(s), a biometric reader(s), and/or the like (not shown). In some variations, the display may include an interactive user interface (e.g., a touch screen) and be configured to transmit a set of commands (e.g., pause, resume, and/or the like) to the light source 424. Additionally, in some variations, the system 400 may include or be communicatively coupled to one or more storage devices (e.g., local or remote memory device(s)).

In some embodiments, the processing device 402 may alternatively or further include additional systems when one or more of the optical sensors may be used for multi-dimensional sensing to detect multiple physical signals, such as temperature and pressure (e.g., to detect multiple different physical signals substantially simultaneously in real-time or near real-time). The measurement signals indicative of physical signals (e.g., temperature information and pressure information) may be determined and then transmitted, for example, to the display or another output device 412 for real-time monitoring or other data related to the measurement region.

The optical sub-system 404 includes the light source control unit 418, the light source 424, optical devices (ODs) 426A, 426B and 426C, and the light receiving device 420. The light source control unit 418 is configured to interface with and control the light source 424 to control the production of an initial optical signal 428. The light source 424 may generate a continuous wave (CW) or pulsed light emission (stimulated emission, spontaneous emission, and/or the like). The initial optical signal 428 may include coherent light, e.g., laser light, provided in one or more modes and at one or more frequencies. The initial optical signal 428 may be of a single frequency/wavelength, a selection of frequencies/wavelengths, and/or a broadband light source. Thus, light source 424 may include a laser array configured to produce laser light in one or more modes and at one or more frequencies. Additionally, the polarization of the supplied light may be controlled to optimize the detected signal levels according to application requirements. The polarization state of light can be controlled to be linearly polarized at certain angles or to be circularly polarized. Linearly polarized light will respond optimally to a certain input ultrasound direction, and circularly polarized light will respond to ultrasound from all directions. The polarization of light can be defined from the laser source output, and the output polarization state can be controlled by an in-line fiber polarizer, a paddle fiber polarization controller, an in-line fiber polarization controller, or other types of polarization controller.

The optical devices 426A, 426B, and 426C may be configured to manipulate or influence the initial optical signal 428 received at the optical sensors 408. The initial optical signal 428 may be provided at a plurality of wavelengths or across a spectrum of wavelengths. The optical device 426A may include, for example, a wavelength division multiplexing (WDM) device configured to multiplex multiple frequencies of initial optical signal 428 provided by the light source 424 for simultaneous transmission over the optical waveguides 430 that direct the initial optical signal 428 to the optical sensors 408. The light source transmits the initial optical signal which passes through a wavelength division multiplexing device (WDM) 426A to the optical device 426B. The optical device 426B may include a WDM device configured to de-multiplex the initial optical signal 428 provided to the optical sensors 408 and subsequently outputs light of a different wavelength. Thus, optical device 426B may be similar to the wavelength division demultiplexers 22/122 of the previous embodiments. Optical device 426B is in optical communication with optical device 426A for dividing the initial optical signal into optical signals each having one of the wavelengths associated therewith and combining the returned optical signals from the optical sensors, which is then directed though an optical device 426C which may include a WDM device (similar to WDM 24/124 of the previous embodiments), to the light receiving device 420.

The initial optical signal 428 is received by the optical sensors 408 and returned through one or more optical waveguides 430 to the optical device 426B, which may be further configured to multiplex the returned optical signal 432 (if required) for transmission to the light receiving device 420. The returned optical signal 432 may be directed by the optical device 426B through and towards the optical device 426C, which may be a WDM device configured to de-multiplex the returned optical signal 432 for reception by the light receiving device 420. The light receiving device 420, which may be a photodetector array, for example, may be in optical communication with optical device 426C for receiving the individual wavelength components of the returned optical signal, such that detected phase shifts or other changes in the individual wavelength components are indicative of sensed acoustic signals.

It will be understood that, in embodiments that do not require frequency multiplexing/demultiplexing of the initial optical signal 428 and the returned optical signal 432, the optical devices 426A and 426B may not be required. The light receiving device 420 may include any suitable device configured to detect incident light, including, for example, a photodetector. The light receiving device 420 may further include, but is not limited to, a photodiode. The light receiving device 420 may be in optical communication with the optical device 426C (e.g., a wavelength division multiplexing splitter) for receiving the individual wavelength components of the returned optical signal 432, such that detected phase shifts, changes in polarization, or other changes in the individual wavelength components are indicative of sensed acoustic signals. The changes in the returned optical signal 432 may be converted (e.g., by the processing unit 414 and/or by additional optical components such as polarization sensitive couplers and/or frequency shifters) into data representative of sensed acoustic signals (which may be further used, e.g., to generate data representative of the tissue/anatomical structure of the medium being insonified or physical signals being measured).

In embodiments, the initial optical signal 428 and returned optical signal 432 signals may undergo pre-processing, beamforming and post-processing, as described in the following applications which disclose various methods for ultrasound beamforming and image processing, each of which is hereby incorporated by reference: U.S. application Ser. No. 18/032,953, titled “Image Compounding for Mixed Transducer Arrays”; U.S. application Ser. No. 18/025,081 titled “Synthetic Aperture Imaging Systems and Methods Using Mixed Arrays”; U.S. application Ser. No. 18/901,073 titled “Acousto-Optic Harmonic Imaging with Optical Sensors”; U.S. patent application Ser. No. 18/280,200 titled “Acoustic Imaging and Measurements Using Windowed Nonlinear Frequency Modulation Chirp”, International Patent Application No. PCT/US2022/077762 titled “Ultrasound Beacon Visualization with Optical Sensors”; and International Patent Application No. PCT/US2022/041252 titled “Multi-Dimensional Signal Detection with Optical Sensors”.

In some embodiments, the output device 412 may further include additional systems, such as a medical procedure or diagnostic system that is configured to use the data that is output. For example, output device 412 may include an endoscopy system, a laparoscopic system, a robotic surgical system, neurosurgical system and additionally may include an interoperative ultrasound imaging system.

It will be understood that the configuration of the optical acoustic sensor system 400 as illustrated in FIG. 5 is provided by way of example. Different configurations may be employed without departing from the scope of this disclosure. For example, different arrangements of optical devices 426A, 426B and 426C, and different numbers and arrangements of optical sensors 408 may be employed. In embodiments, the light source control unit 418 and the acoustic control unit 422 may be incorporated or integrated within the processing system 402. Additional combinations of the components of the optical acoustic sensor system 400 may be selected as appropriate to achieve the functionality as described herein.

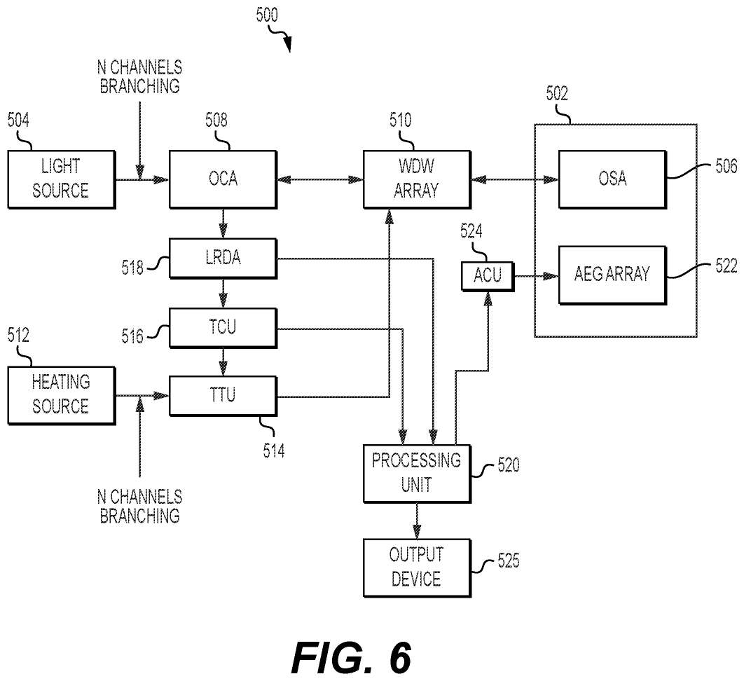

FIG. 6 illustrates an optical acoustic sensor system 500 for use with a mixed sensor array. The optical acoustic sensor system 500 includes components, devices, hardware, and software to facilitate the use of a mixed sensor array 502. Certain aspects of the optical acoustic sensor system 500 are similar to that of optical acoustic sensor system 400 of FIG. 5 and are not repeated. Aspects that differ are described below.

The optical acoustic sensor system includes a light source 504, including a single laser or several lasers (e.g., to boost power) operating at the same wavelength. The initial optical signal from the light source 504 is separated into a number of channels that corresponds to the number of fiber optic sensors in the optical sensor array (OSA) 506. The initial optical signal passes through an optical circulator array (OCA) 508, including a number of circulators that correspond to the number of fiber optic sensors, with each signal being directed to a WDM unit from a WDM array 510.

The optical acoustic sensor system also includes a heating source 512, including a single laser or several lasers (e.g., to boost power) operating at the same wavelength. The heating source 512 operates at a frequency configured for thermal absorption by the fiber optical sensors of the optical sensor array 506, as discussed herein. The initial thermo-optical signal from the heating source 512 is separated into a number of channels that correspond to the number of fiber optic sensors in the optical sensor array 506. The initial thermo-optical signal(s) pass through a thermal tuning unit 514 that operates to adjust the intensity of each thermo-optical signal to tune the individual optical sensors of the optical sensor array 506. The thermal tuning unit may operate, for example, by use of an electrical variable optical attenuator. The resultant tuned thermo-optical signals are provided to the WDM array 510 to be multiplexed with a corresponding initial optical signal and provided to the appropriate optical sensor of the optical sensor array 506. The thermal tuning unit (TTU) 514 is controlled by the thermal control unit (TCU) 516 which receives input from the light receiving device array (LRDA) 518. Input from the light receiving device array 518 is used in a feedback loop to control the heating (and thus the thermal tuning properties) of each fiber optic sensor of the optical sensor array 506 individually. The thermal tuning process is described above and may be used to tune the individual fiber optic sensors of the optical sensor array 506 to be sensitive to the same operating laser frequency.

Additional features of the optical acoustic sensor system 500 are similar to those of optical acoustic sensor system 400. The returned optical signals are filtered from the thermo-optical signals and passed through the circulator array 508 where they are directed to the light receiving device array 518. Alternatively, the light receiving device array 518 may be selected as a device that is relatively insensitive to the wavelength of the thermo-optical signals, allowing receipt of these signals without unduly affecting the temperature of the light receiving device array 518. The light receiving device array 518 is configured to receive the multiple returned optical signals (e.g., via individual light receiving devices of the array, where each light receiving device corresponds to one of the channels into which the initial optical signal is separated) and provide information and data thereof to the processing unit 520. The individual light receiving devices may be, for example, individual photodetectors.

The processing unit 520 further communicates with the AEG array 522 (for generating acoustic energy) via the acoustic control unit (ACU) 524. Information from the AEG array 522 and the optical sensor array 506 are used by the processing unit 520 in acoustic environment determinations, including, e.g., imaging. In addition, the processing unit 520 may also receive output from the thermal tuning control unit 516 for use in interpreting the returned optical signals. Acoustic determination information may be output via the output device 525, which may be, for example, a display, another medical system, etc. The optical acoustic sensor system 500 significantly reduces the required number of lasers for the light source 504 by splitting the optical signal from a single light source 504 into multiple channels. This may reduce the cost, size, and power consumption of the system 400.

It is to be understood that the optical sensor circuit and the optical sensing method are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. An optical sensor circuit, comprising:
an optical input port for receiving a plurality of input optical signals within a single input channel, wherein each of the input optical signals has a unique wavelength associated therewith;
a wavelength-division demultiplexer coupled to the optical input port to demultiplex the plurality of input optical signals;
a plurality of optical sensors coupled to the wavelength-division demultiplexer for respectively receiving the plurality of input optical signals and outputting a corresponding plurality of output optical signals;
a wavelength-division multiplexer coupled to the plurality of optical sensors to multiplex the plurality of output optical signals into a single output channel; and
an optical output port coupled to the wavelength-division multiplexer for outputting the plurality of output optical signals in the single output channel.

2. The optical sensor circuit as recited in claim 1, wherein each of the output optical signals has a unique wavelength associated therewith matching the wavelength of a corresponding one of the input optical signals.

3. The optical sensor circuit as recited in claim 1, wherein each of the optical sensors comprises an opto-acoustic sensor.

4. The optical sensor circuit as recited in claim 1, wherein the plurality of optical sensors comprise a fiber optical sensor array.

5. The optical sensor circuit as recited in claim 1, further comprising an acoustic transducer, wherein the plurality of optical sensors and the acoustic transducer are mounted together in a mixed sensor transducer probe.

6. An optical sensor circuit, comprising:
a plurality of optical input ports for respectively receiving a plurality of input channels, wherein each of the input channels carries a plurality of input optical signals, wherein each of the input optical signals within each of the input channels has a unique wavelength associated therewith;
a power splitter for splitting each of the input channels into a plurality of sub-channels carrying a plurality of input optical sub-signals;
a wavelength-division demultiplexer coupled to the power splitter to demultiplex each of the plurality of input optical sub-signals;
a plurality of optical sensors coupled to the wavelength-division demultiplexer for respectively receiving the plurality of input optical sub-signals and outputting a corresponding plurality of output optical signals;
a wavelength-division multiplexer coupled to the plurality of optical sensors to multiplex the plurality of output optical signals into a plurality of output channels; and
a plurality of optical output ports coupled to the wavelength-division multiplexer for outputting the plurality of output optical signals in the plurality of output channels.

7. The optical sensor circuit as recited in claim 6, wherein each of the output optical signals has a unique wavelength associated therewith matching the wavelength of a corresponding one of the input optical sub-signals.

8. The optical sensor circuit as recited in claim 6, wherein a total number of the plurality of output channels is equal to a total number of the output optical signals divided by a total number of the input channels.

9. The optical sensor circuit as recited in claim 6, wherein each of the optical sensors comprises an opto-acoustic sensor.

10. The optical sensor circuit as recited in claim 6, wherein the plurality of optical sensors comprise a fiber optical sensor array.

11. The optical sensor circuit as recited in claim 6, further comprising an acoustic transducer, wherein the optical sensors and the acoustic transducer are mounted together in a mixed sensor transducer probe.

12. An optical sensing method, comprising:
receiving a plurality of input optical signals within a single input channel, wherein each of the input optical signals has a unique wavelength associated therewith;
demultiplexing the plurality of input optical signals;
respectively inputting the demultiplexed plurality of input optical signals into a plurality of optical sensors; and
multiplexing a plurality of output optical signals respectively output by the plurality of optical sensors into a single output channel.

13. The optical sensing method as recited in claim 12, wherein each of the output optical signals has a unique wavelength associated therewith matching the wavelength of a corresponding one of the input optical signals.

14. An optical sensing method, comprising:
receiving a plurality of input channels, wherein each of the input channels carries a plurality of input optical signals, wherein each of the input optical signals within each of the input channels has a unique wavelength associated therewith;
power splitting each of the input channels into a plurality of sub-channels carrying a plurality of input optical sub-signals;
demultiplexing each of the plurality of input optical sub-signals;
respectively inputting the demultiplexed plurality of input optical sub-signals into a plurality of optical sensors; and
multiplexing a plurality of output optical signals respectively output by the plurality of optical sensors into a plurality of output channels.

15. The optical sensing method as recited in claim 14, wherein each of the output optical signals has a unique wavelength associated therewith matching the wavelength of a corresponding one of the input optical sub-signals.

16. The optical sensing method as recited in claim 14, wherein a total number of the plurality of output channels is equal to a total number of the output optical signals divided by a total number of the input channels.

17. An optical acoustic sensor system, comprising:
an acoustic transducer for delivering an acoustic signal to a sample to be sensed;
a light source for generating a plurality of input optical signals, wherein each of the input optical signals has a unique wavelength associated therewith;
a wavelength-division demultiplexer coupled to the light source to demultiplex the plurality of input optical signals;
a plurality of optical sensors for sensing the sample, the plurality of optical sensors being coupled to the wavelength-division demultiplexer for respectively receiving the plurality of input optical signals and outputting a corresponding plurality of output optical signals;
a wavelength-division multiplexer coupled to the plurality of optical sensors to multiplex the plurality of output optical signals into a single output channel; and
means for processing the single output channel to produce an image of the sample.

18. The optical acoustic sensor system as recited in claim 17, wherein each of the output optical signals has a unique wavelength associated therewith matching the wavelength of a corresponding one of the input optical signals.

19. The optical acoustic sensor system as recited in claim 17, wherein each of the optical sensors comprises an opto-acoustic sensor.

20. The optical acoustic sensor system as recited in claim 17, wherein the optical sensors comprise a fiber optical sensor array.

21. The optical acoustic sensor system as recited in claim 17, wherein the optical sensors and the acoustic transducer are mounted together in a mixed sensor transducer probe.

22. The optical acoustic sensor system as recited in claim 20, further comprising a heating source for selectively heating individual ones of the optical sensors of the optical sensor array for tuning thereof.

23. The optical acoustic sensor system as recited in claim 17, further comprising means for processing the single output channel to produce data representative of multi-dimensional sensing.

24. An optical sensor circuit, comprising:
an optical input port for receiving a plurality of input optical signals within a single input channel, wherein each of the input optical signals has a unique wavelength associated therewith;
a plurality of optical sensors for respectively receiving the plurality of input optical signals and outputting a plurality of output optical signals;
a wavelength-division multiplexer for receiving individual wavelength components of the plurality of output optical signals;
an optical output port coupled to the wavelength-division multiplexer for outputting the plurality of output optical signals in the single output channel; and
means for processing the single output channel to produce data representative of sensed physical parameters.
